(19) United States
(12) Patent Application Publication
(10) Pub. No.: US 2002/0168660 A1
(43) Pub. Date: Nov. 14, 2002

(54) Stem cell self-renewal and lineage commitment

(75) Inventors: Chang-Zheng Chen, Brookline, MA (US); Harvey F. Lodish, Brookline, MA (US)

Correspondence Address: HAMILTON, BROOK, SMITH & REYNOLDS, P.C., 530 VIRGINIA ROAD, P.O. BOX 9133, CONCORD, MA, 01742-9133, US

(73) Assignee: Whitehead Institute for Biomedical Research, Cambridge, MA (US)

(21) Appl. No.: 10/077,178

(22) Filed: Feb. 15, 2002

Related U.S. Application Data
(60) Provisional application No. 60/269,060, filed on Feb. 15, 2001.

(51) Int. Cl. C12Q 1/68; C12N 5/08; C12N 5/06

(52) U.S. Cl. 435/6; 435/372; 435/354

(57) ABSTRACT

Methods of marking pluripotent cells, such as stem cells, particularly hematopoietic stem cells; methods of detecting/identifying, enriching, selecting and monitoring pluripotent cells (stem cells); DNA constructs useful in the methods, stem cells, such as hematopoietic stem cells, identified by the method, as well as lineage-specific cells identified by the method; and uses for the cells are subjects of this invention.

### RELATED APPLICATION(S)

This application claims the benefit of U.S. Provisional Application No. 60/269,060, entitled “Stem Cell Self-Renewal and Lineage Commitment”, filed on Feb. 15, 2001.

The entire teachings of the above application are incorporated herein by reference.

### FUNDING

Work described herein was funded, in whole or in part, by National Institutes of Health Grant P01 HL 32262. The United States Government has certain rights in the invention.

### BACKGROUND OF THE INVENTION

In mammals, hematopoietic stem cells (HSCs) are responsible for the daily production of millions of mature cells of all blood lineages throughout adult life. The ability of HSC to differentiate into all blood cell types, together with their ability to self-renew, constitutes the unique biological function of HSC (Morrison, S. J., et al., Cell. 88:287-98 (1997)). Abnormalities in this development program lead to blood cell diseases including leukemia. Therefore, understanding the molecular mechanisms that control HSC self-renewal processes and the differentiation decisions is not only of biologic significance, but also has implications in bone marrow transplantation, gene therapy, and disease pathogenesis, such as leukemia.

Much effort in stem cell research to date has been devoted to establish a culture system to maintain and expand this rare population cells in vitro. This is not only the essential first step to understand the cellular and molecular mechanisms that govern HSC self-renewal and lineage commitment, but also has a wide range of clinical applications including leukemia therapy and gene therapy. Various stromal lines, or defined cytokines, or combinations of both were tested to recapitulate hematopoietic stem cells self-renewal and differentiation in culture systems (Moore, K. A., et al., Blood, 89:4337-47 (1997)). In many cases, transplantable HSCs can only be maintained on stromal layers for a limited period of time. However, most attempts to identify culture conditions to support a net expansion of transplantable HSCs had limited success, in spite of considerable success with the expansion of later types of progenitor cells (Audet, J., et al., Current Opinion in Biotechnology, 9:146-51 (1998)). The key extracellular and intracellular signals that govern stem cell renewal and differentiation remain elusive (Lemischka, I., et al., Annals of New York Academy of Sciences, 872:274-87; discussion 287-8 (1999)).

Current functional assays for hematopoietic stem cells, including mice repopulating assays and other surrogate in vitro assays such as cobble stone area assay and LTC-IC, are all long-term assays (Jordan, C. T., et al., Experimental Hematology, 23:1011-5 (1995); Ploemacher, R. E., et al., Blood, 74:2755-63 (1989); Szilvassy, S. J., et al, Proceedings of the National Academy of Sciences of the United States of America, 87:8736-40, (1990); Eaves, C. J., et al., Annals of the New York Academy of Sciences, 628:298-306 (1991)). By nature, they are quite tedious and time consuming, and cannot be used in real-time to monitor HSC activity. This significantly restricts the ability to detect, select and monitor HSCs in vitro and in vivo, which is essential for understanding the cellular and molecular mechanisms that mediate HSCs self-renewal and lineage commitment. Real-time functional markers for HSCs, if available, would overcome the limitations of these assays, allow analysis of factors that could control HSC fate and, thus, make it possible to monitor HSC, as well as identify and isolate them.

### SUMMARY OF THE INVENTION

Described herein is a new approach to mark pluripotent cells, such as stem cells (e.g., hematopoietic stem cells) by targeting reporter genes into loci that are functionally specific and important for hematopoietic stem cell activity (e.g., self-renewal or lineage commitment). Combinations of targeted markers have been used to provide physical and functional identities for hematopoietic stem calls. Marked hematopoietic stem cells will greatly facilitate identification, enrichment, selection and monitoring of hematopoietic stem cells in vitro and in vivo. Methods of marking such pluripotent cells; methods of detecting/identifying, enriching, selecting and monitoring pluripotent cells (stem cells); DNA constructs useful in the methods; stem cells, such as hematopoietic stem cells, identified by the method, as well as lineage-specific cells identified by the method; and uses for the cells are subjects of this invention.

In one embodiment, two loci, Stem Cell Leukemia (SCL) and Ikaros, are used to define hematopoietic stem cells functionally and phenotypically. SCL and Ikaros, which play important roles in hematopoiesis in mice, are co-expressed in hematopoietic stem cells (HSCs), but not in the same differentiated lineages. SCL and Ikaros are structurally and functionally conserved between humans and mice. HSCs have been selectively marked by targeting reporter DNAs into the SCL and Ikaros loci, respectively. Since only the HSCs express both reporters, HSCs can be identified in vivo by selective culturing or histological staining, optionally enriched, such as by FACS sorting, and selectively cultured in order to further select for or purify hematopoietic stem cells. In one embodiment, hematopoietic stem cells are selectively marked by targeting two constructs—huCD4/IRES/puro and βneo (lacZneo)—into SCL and Ikaros, respectively. Since only the hematopoietic stem cells express both reporters, they can be identified based on the presence of both markers; optionally, enriched (e.g., by FACS sorting); and selectively cultured in the presence of G418 and puromycin. As a result, hematopoietic stem cells are identified and isolated. This provides a powerful tool to explore, for example, the conditions to expand hematopoietic stem cells in vitro, and to identify signal molecules that control hematopoietic stem cell self-renewal and lineage commitment, which may provide improvements in current bone marrow transplantation and leukemia therapy.

One embodiment of the present invention is a method of marking or identifying hematopoietic stem cells. The method comprises: (a) introducing (targeting) two different reporter DNAs (e.g., two genes that confer resistance to two different antibiotics; two “sets” of different reporter DNAs, each of which includes a gene that confers resistance to an antibiotic and DNA that encodes a marker for histological staining; two genes that encode different fluorescent proteins wherein each fluorescent proteins fluoresce at a different wavelength) into two different functionally important genomic loci of hematopoietic stem cells in such a manner that expression of the reporter DNA is driven by the promoter of the genomic loci into which the reporter DNA is targeted, thereby producing a population of cells that comprises successfully targeted hematopoietic stem cells, which are hematopoietic stem calls having two different reporter DNAs incorporated into two different functionally important genomic loci and other cells; (b) subjecting the population produced in (a) to conditions under which successfully targeted hematopoietic stem cells can be differentiated from the other cells. In one embodiment, the population produced in (a) is subjected to conditions under which successfully targeted hematopoietic stem cells survive and the other cells in the population do not, thereby identifying hematopoietic stem cells. In a particular embodiment, the two different reporter DNAs are DNAs that confer resistance to two different antibiotics, such as G418 and puromycin, and the population of cells is cultured in the presence of the two drugs, which results in survival of only cells that were successfully targeted with both reporter DNAs. Different functionally important genomic loci include, for example, the SCL locus, the Ikaros locus, the LMO2 locus, the LY1 locus, the c-kit locus and the Notch-1 locus. As a result, hematopoietic stem cells are identified; they can be removed from the mixture in which they are present, using known methods.

In a particular embodiment, the present invention relates to a method of identifying hematopoietic stem cells, comprising targeting a first reporter DNA into a Stem Cell Leukemia (SCL) locus and a second reporter DNA which is different from the first reporter DNA into an Ikaros locus of hematopoietic stem cells in such a manner that expression of the first reporter DNA is driven by the promoter of the SCL locus and the expression of the second reporter DNA is driven by the promoter of the Ikaros locus. A population comprising successfully targeted hematopoietic stem cells and other cells are thereby produced. The population of cells produced in (a) is then subjected to conditions under which successfully targeted hematopoietic stem cells survive and the other cells in the population do not survive, thereby identifying hematopoietic stem cells.

A further embodiment of the method of identifying hematopoietic stem cells comprises: (a) introducing (targeting) two different reporter DNAs (e.g., two genes that confer resistance to two different antibiotics; two genes that confer resistance to two different antibiotics, each in combination with DNA encoding a marker for histological staining) into two different functionally important genomic loci in such a manner that expression of the reporter DNA is driven by the promoter of the genomic loci into which the reporter DNA is targeted, thereby producing a population of cells that comprises successfully targeted hematopoietic stem cells, which are hematopoietic stem cells having two different reporter DNAs incorporated into two different functionally important genomic loci and other cells; (b) subjecting the population produced in (a) to conditions under which successfully targeted hematopoietic stem cells survive and the other cells in the population do not, thereby identifying hematopoietic stem cells, which are present in a mixture; (c) enriching the mixture of (b) for hematopoietic stem cells, thereby producing an enriched population of hematopoietic stem cells; and (d) subjecting the enriched population of stem cells to conditions under which successfully targeted hematopoietic stem cells survive and other cells do not, thereby identifying hematopoietic stem cells. In a particular embodiment, the two different reporter DNAs are DNAs that confer resistance to two different antibiotics, such as G418 and puromycin, and the population of cells is cultured in the presence of the two drugs, which results in survival of only cells that were successfully targeted with both reporter DNAs. The two different functionally important genomic loci can be, for example, the SCL locus and the Ikaros locus; the LMO2 locus and the LY1 locus; and the c-kit locus and the Notch-1 locus or varying combinations thereof. As a result, hematopoietic stem cells are identified; they can be removed from the mixture in which they are present, using known methods.

In an additional embodiment of identifying hematopoietic stem cells, the two different reporter DNAs targeted into two different functionally important loci in the cells comprise, in addition to DNA encoding resistance to antibiotics, DNA encoding a marker for histological staining. This embodiment comprises: (a) introducing (targeting) two different reporter DNAs (two genes that confer resistance to two different antibiotics, each in combination with DNA encoding a marker for histological staining) into two different functionally important genomic loci in such a manner that expression of the reporter DNA is driven by the promoter of the genomic loci into which the reporter DNA is targeted, thereby producing a population of cells that comprises successfully targeted hematopoietic stem cells, which are hematopoietic stem cells having two different reporter DNAs incorporated into two different functionally important genomic loci and other cells; (b) subjecting the population produced in (a) to histological staining, whereby cells that express the marker for histological staining are identified; (c) enriching the population of (b) for hematopoietic stem cells, thereby producing an enriched population of hematopoietic stem cells; and (d) subjecting the enriched population of stem cells to conditions under which successfully targeted hematopoietic stem cells survive and other cells do not, thereby identifying hematopoietic stem cells. In a particular embodiment, the two different reporter DNAs are DNAs that confer resistance to two different antibiotics, such as G418 and puromycin, and DNAs that encode a histological marker, such as huCD4 or LacZ (beta-galactosidase), can be stained by 4-chloro-5-bromo-3indolyl-beta-galactoside (X-gal). For histological staining, the population is stained with anti human CD4 antibody or X-gal, respectively. CD4 and X-gal staining makes it possible to reveal details of (a) the development of hematopoietic stem cells and differentiated myeloid or lymphoid cell lineages in a developing mouse embryo; (b) the micro-environment of hematopoietic stem cells and differentiated myeloid or lymphoid cell lineages in a developing animal or an adult animal; and (c) the migration of hematopoietic stem cells and differentiated myeloid or lymphoid cell lineages in a developing animal or an adult animal. This may provide important information about in vivo mechanisms that regulate hematopoietic stem cell self-renewal and lineage commitment.

In an additional embodiment, βuCD4 and lacZ can also be used as markers to enrich hematopoietic stem cells by FACS sorting. The population can be enriched for hematopoietic stem cells, such as by FACS, and the resulting enriched population can then be cultured in the presence of the two drugs, which results in survival of only cells that were successfully targeted with both reporter DNAs. As a result, hematopoietic stem cells are identified; they can be removed from the mixture in which they are present, using known methods.

Another embodiment of the invention is a real-time surrogate assay for hematopoietic stem cell activity, which is possible because only hematopoietic stem cells (and not differentiated lineage cells) express both the huCD4 and lacZ reporter genes.

In a further embodiment, embryonic stem cells derived from double transgenic animals, such as mice, in which the SCL and the Ikaros loci have been targeted with reporter DNAs, can be used to study the conditions needed to cause embryonic stem cells to differentiate into hematopoietic stem cells or a specific lineage cell type.

SCL and Ikaros are structurally and functionally conserved between humans and mice and, therefore, it is possible, using known methods, to produce human embryonic stem cells with reporter DNAs targeted into the SCL and Ikaros loci. These reporters can be used to study the conditions that cause or result in differentiation of human embryonic stem cells into hematopoietic stem cells or a specific lineage cell type.

Also encompassed by the present invention are hematopoietic stem cells isolated by the methods. In one embodiment, the present invention relates to isolated hematopoietic stem cells comprising two different reporter DNAs which are present in two different functionally important genomic loci of the hematopoietic stem cells, wherein expression of the reporter DNAs is driven by the promoters of the genomic loci into which the reporter DNAs are targeted.

In another embodiment, the present invention relates to isolated hematopoietic stem cells comprising a first reporter DNA which is present in a Stem Cell Leukemia (SCL) genomic locus of the hematopoietic stem cells and a second reporter DNA which is different from the first reporter DNA and which is present in an Ikaros genomic locus of the hematopoietic stem cells, wherein expression of the first reporter DNA is driven by the promoter of the SCL locus and expression of the second reporter DNA is driven by the promoter of the Ikaros locus.

### BRIEF DESCRIPTION OF THE DRAWINGS

FIG. 1 is a schematic diagram of hematopoietic stem cell differentiation model and the SCL/Ikaros expression profile in HSCs and differentiated lineages. According to this model, the SCL and Ikaros positive cells should contain HSCs. Therefore, HSCs can be selectively marked with huCD4/IRES/Puro and lacZneo reporter genes targeted into SCL and Ikaros, respectively. These genetic markers can be utilized to enrich HSCs by FACs and to select HSCs with drug in an in vitro culture system depicted in the diagram.

FIGS. 2a and 2b are schematic representation of transgenic constructs. Both transgenic constructs were generated by homologous recombination in Bacterial Artificial Chromosome (BAC) DNAs (Yang, X. W., et al., Nature Genetics, 22:327-35(1999); Yang, X. W., et al., Nature Biotechnology, 15:859-65 (1997)).

FIG. 2a is a schematic representation of the SCL transgenic construct. Reporter gene huCD4/IRES/puro was targeted into the SCL locus by deleting exon 4 and exon 5.

FIG. 2b is a schematic representation of the Ikaros transgenic construct. Reporter gene Beta-geo (lacZ-neo) was targeted into the Ikaros locus by fusing to exon 3, deleting exon 4 and part of exon 3.

FIG. 3a is a schematic diagram of retroviral expression cloning.

FIG. 3b is a schematic representation of identification of positive regulators of SCL and Ikaros expression.

FIG. 3c is a schematic representation of identification of negative regulators of SCL and Ikaros expression.

FIG. 4 is a schematic diagram of “Referenced PCR Amplification” and its application in DNA microanalysis.

FIG. 5 shows that reporter constructs lacZ/neo/polyA and CD4 /IRES/puro/polyA.

FIG. 6A show that hematopoietic stem cells can be further enriched by using SCL-bgeo transgenic marker in combination with Hoechst 33342 dye and lineage markers; SCL+“SP” account for about 17-20% of linearized negative “SP” cell.

FIG. 6B shows that lineage negative bone marrow (BM) cells can be categorized into four putative functional cell populations: SCL+“SP”, SCL−“SP”, SCL+“nonSP”, SCL−“nonSP” which presumably represents HSCs, other tissue specific stem cells (TSCs), early myeloid progenitors, early lymphoid progenitors and other non-myeloid cells, respectively.

### DETAILED DESCRIPTION OF THE INVENTION

Described herein are methods of detecting/identifying, enriching, selecting and monitoring stem cells (pluripotent cells), such as hematopoietic stem cells, in which at least one (one or more) reporter (or marker) DNA(s) (e.g., gene(s)) is introduced, by targeting, into at least one locus (one or more loci) and usually at least two loci in genomic DNA of the pluripotent cells that are functionally important for self-renewal and/or lineage commitment of the pluripotent cells. Such loci, referred to as functionally important genomic loci, are co-expressed in the stem cell, but both are not expressed in the differentiated cell lineages that result from the stem cell. The genes at the loci are essential or important for the development and differentiation of hematopoietic stem cells. Coexpression facilitates identification of stem cells, such as hematopoietic stem cells, and differential expression in lineage cells facilitates identification of the lineage cells. For convenience, the term reporter DNA is used to refer to DNA that encodes a product that acts as a reporter or marker or itself acts as a reporter or marker (e.g., antibiotic resistance; substances, such as proteins or enzymatic or metabolic substrates). Reporter DNA can comprise, in effect, two sets of reporters, each of which enables identification of hematopoietic stem cells and cells of the myeloid pathway and cells of the lymphoid pathway. As described in greater detail below, reporter DNA can be, for example, DNA that encodes antibiotic resistance. It can additionally include a second DNA that encodes a product, such as a protein or an enzymatic or metabolic substrate, that permits histological screening, such as with an appropriate antibody or enzymatic reaction, that identifies hematopoietic stem cells. Two such sets of reporter DNAs are described herein: one, designated huCD4/IRES/puro, that permits staining of hematopoietic cells with anti-human CD4 antibodies, as well as selective culturing in the presence of puromycin and one, designated BetaGeo or bgeo (lacZneo), that permits staining with X-gal or FDG, a fluorescent substrate of LacZ and selective culturing in the presence of G418. All reporter DNAs described herein can be introduced into any functionally important genomic locus. The description of reporter DNAs provided herein, in which they are discussed in the context of a particular locus, are simply for purposes of illustration.

In a preferred embodiment, two different reporter DNAs (two different sets of reporter DNAs) are targeted into two different functionally important genomic loci of a type of stem cell (e.g., hematopoietic stem cells) of interest. Targeting of the reporter DNAs into the loci results in their being under the control of the regulatory sequences (promoter) of the endogenous targeted gene and, as a result, they are expressed along with the endogenous gene. Both loci are present/co-expressed in the pluripotent (stem) cells, but both are not expressed in the differentiated lineages that develop from the stem cells as they differentiate. One locus is expressed in one cell lineage and the second locus is expressed in the second cell lineage into which the stem cell differentiates. As a result, stem cells can be identified by selecting for cells that express both reporter DNAs. This can be done, for example, by subjecting a population of cells that has been targeted for introduction of two reporter different DNAs (e.g., two different antibiotic resistance genes) into two different functionally important genomic loci to conditions under which only cells that have been successfully targeted can survive (e.g., by culturing cells in medium that contains the two antibiotics, against which the genes confer resistance). Alternatively, they can be identified by means of histological staining, as described herein. In this instance, also targeted into the two loci, along with the reporter gene that confers drug resistance is a reporter DNA that encodes a marker for histological staining. The population of cells that has been subjected to the targeting procedures is a mixed population, comprising stem cells that have been successfully targeted with the two reporter DNAs, stem cells that have not been successfully targeted for the reporter DNAs and other cell types. Stem cells are, thus, identified and isolated and can be further assessed or expanded. Alternatively, they can be maintained under conditions appropriate for differentiation and subjected to further conditions that permit stem cells that contain one reporter DNA to survive but result in the death of those that contain the second (different) reporter DNA. For example, in the case in which the reporter DNAs confer antibiotic resistance on successfully targeted stem cells, part of the population can be cultured in the presence of one of the antibiotics and part in the presence of the second antibiotic, thus making it possible to distinguish between the two cell types. This is further illustrated below with specific reference to hematopoietic stem cells and the myeloid and lymphoid lineage cells that result from differentiation of the stem cells. However, this method is useful to identify, select, enrich and/or monitor any type of stem cell and the lineages into which it differentiates. In those instances in which only a small percentage of SCL and Ikaros positive cells are stem cells of interest, an additional reporter DNA targeted into an additional locus may be needed to specify (identify) the stem cell population.

In a specific embodiment of the present invention, two or more different reporter DNAs are targeted into two or more different genomic loci of hematopoietic stem cells that are functionally important for HSC self-renewal and/or lineage commitment. Both loci are expressed in the hematopoietic stem cells, but not in the differentiated lineages (e.g., the myeloid lineage and the lymphoid pathway). Because the loci are co-expressed only in hematopoietic stem cells, both reporter DNAs are co-expressed only in hematopoietic stem cells (and not in the differentiated lineage cells) As a result, hematopoietic stem cells can be identified in vivo (e.g., by histological staining). They can, optionally, be enriched (e.g., by FACS sorting) or selectively cultured (e.g., in the presence of agents, such as antibiotics, that kill or inhibit the growth of cells that do not contain both targeted reporter DNAs). The resulting isolated hematopoietic stem cells can be further assessed, such as by determining the conditions appropriate for in vitro expansion of such cells, thus making it possible to produce populations of stem cells useful clinically and for research purposes. In addition, the resulting cells can be used to identify signal molecules that control (enhance, block, maintain) hematopoietic stem cell self renewal and lineage commitment. Information gained in this way can be used to assess and hopefully improve current bone marrow transplantation leukemia therapy. Alternatively, the resulting hematopoietic stem cells can be permitted to differentiate and the resulting myeloid and lymphoid cell lineages then identified, enriched, selected and/or isolated by means of the different reporter DNAs targeted into lineage-specific loci in the stem cell. For example, stem cells maintained under conditions that permit them to undergo differentiation can be subjected to further conditions that permit stem cells that contain one reporter DNA to survive, but result in the death of those that contain the second (different) reporter DNA. For example, in the case in which the reporter DNAs confer antibiotic resistance on successfully targeted stem cells, part of the population can be cultured in the presence of one of the antibiotics and part in the presence of the second antibiotic, thus making it possible to distinguish between the two cell types. This can be done, for example, by selectively culturing isolated hematopoietic stem cells, after they have undergone sufficient differentiation, in the presence of agents (e.g., antibiotics) that kill or inhibit the growth of cells that do not contain the targeted DNA that confers resistance on recipient hematopoietic stem cells.

Two different functionally important genomic loci for use in the present invention include, for example, the SCL locus and the Ikaros locus, the LMO2 locus and the LY1 locus, the c-kit locus and the Notch-1 locus and various combinations thereof.

In a specific embodiment, two loci co-expressed in hematopoietic stem cells, but not in myeloid and lymphoid lineage cells, are targeted with two different reporter DNAs. One of the two loci is expressed in myeloid lineage cells and the other is expressed in lymphoid lineage cells. For example, the Stem Cell Leukemia (SCL) locus, which is expressed in myeloid lineage cells but not in lymphoid lineage cells and the Ikaros locus, which is expressed in lymphoid lineage cells but not in myeloid lineage cells can be targeted. In one embodiment, reporter DNAs, which confer antibiotic resistance on successfully targeted cells can be used. For example, as described further herein, resistance to G418 and puromycin can be conferred upon hematopoietic stem cells by introducing constructs that comprise the neo gene and the puromycin resistance gene and target one of the genes into the SCL locus and the other into the Ikaros locus in such a manner that the reporter genes are expressed under the control of the endogenous gene's regulatory sequences. In an embodiment described herein, the neo gene is introduced into the SCL locus and the gene conferring puromycin resistance is introduced into the Ikaros locus. Alternatively, gene conferring puromycin resistance can be targeted into the SCL locus and the neo gene into the Ikaros locus. Hematopoietic stem cells that express both reporters are identified, such as by histological staining or selective culturing in antibiotic-containing medium. The resulting population is generally an enriched population (a mixture) that comprises targeted hematopoietic stem cells co-expressing the two reporter DNAs, since cells that express neither or only one of the reporter genes are eliminated from the culture. Optionally, the hematopoietic stem cell population can be further enriched, using known methods, such as FACS sorting. The resulting enriched hematopoietic stem cell population is then further purified by culturing it in the presence of the agents that kill or inhibit growth of cells that have not been successfully targeted with the two reporter genes. For example, the enriched population is cultured in the presence of G418 and puromycin. The resulting hematopoietic stem cells can be assessed or expanded, using known methods, for the purposes described herein.

Alternatively, hematopoietic stem cells obtained can be used to study the mechanisms that control lineage-specific differentiation. For example, if the CD4/IRES/puro DNA is introduced into the SCL locus and the lacZneo DNA is introduced into the Ikaros locus, cells that differentiate into myeloid cells (enter the myeloid pathway) are SCL⁺/Ikaros⁻ nand those that differentiate into lymphoid cells (enter the lymphoid pathway) are SCL⁻/Ikaros⁺. Therefore, lineage-specific differentiation can be evaluated by means of FACS analysis or drug selection.

In a specific embodiment of the present method, huCD4/IRES/puro and BetaGeo (lacZneo) are targeted into the SCL and Ikaros loci, respectively, of hematopoietic stem cells in such a manner that they are co-expressed in successfully targeted recipient cells. The successfully targeted hematopoietic stem cells are identified using known methods, such as histological staining (e.g., with anti-human CD4 antibody, to identify huCD4-expressing stem cells and FDG, to identify stem cells expressing BetaGeo) or culturing in antibiotic-containing medium (in this example, G418- and puromycin-containing medium). The population of hematopoietic stem cells, which is likely to contain other cell types, is optionally, enriched by FACS sorting and the resulting enriched population of hematopoietic stem cells is selectively cultured in the presence of G418 and puromycin to further enrich the hematopoietic stem cell population. The hematopoietic stem cells thus obtained can be assessed, as described above, to determine conditions appropriate to expand hematopoietic stem cells and to identify signal molecules that control their self-renewal and lineage commitment. Alternatively, as also described above, hematopoietic stem cells obtained in this manner can be maintained under conditions appropriate for differentiation to occur and grown in the presence of one of the antibiotics, in order to identify those cells that enter the myeloid pathway and those that enter the lymphoid pathway. In the former case, the cells are grown in the presence of puromycin and in the former case, in the presence of G418; cells that survive in the presence of puromycin are myeloid pathway cells and those that survive in the presence of G418 are lymphoid pathway cells. Here, too, the cells obtained can be assessed, for example, to determine conditions appropriate to expand the cells and to identify signal molecules and other factors that permit or result in their further differentiation (e.g., common myeloid progenitors (CMPs) can form all cells of granulocyte/macrophage or megakaryocyte/erythrocyte lineages and common lymphoid progenitors (CLPs) can form all cells of the lymphoid lineages.

The hematopoietic stem cells used in and resulting from the methods of the present invention can be from a wide variety of organisms, including vertebrates and non-vertebrates, mammals and non-mammals, including, but not limited to, mice, rats, pigs, dogs, cats, cows, goats, sheep, non-human primates and humans. Similarly, a wide variety of functionally important genomic loci can be the target for introduction of reporter DNAs. SCL and Ikaros are specifically described herein. As described in detail in the example, they both play important roles in mice hematopoiesis, and are co-expressed in hematopoietic stem cells, but are not expressed in the same differentiated lineages. Alternatively, two additional sets of genes, c-kit and Notch-1, and LMO2 and LYL1 can be used to mark hematopoietic stem cells, such as in combination with SCL and Ikaros.

The present invention is illustrated by the following exemplification, which is not intended to be limiting in any way.

Exemplification

The aims of the work described below are as follows:

1. Develop a rationale for marking HSCs by targeting reporters into SCL and Ikaros loci.

2. Target reporters into SCL and Ikaros Loci through gene targeting and BAC-mediated transgenesis.

3. Examine the functional properties of the cells marked by SCL and Ikaros transgenic mice.

4. Utilize other HSC specific genes in combination with SCL or Ikaros to mark HSCs.

5. Explore conditions to control HSCs self-renewal and lineage commitment in vitro.

6. Identify signal molecules that control HSC self-renewal and lineage commitment by retroviral expression cloning.

Experimental Design

1. Rationale for Marking HSCs by Targeting Reporters Into SCL and Ikaros loci.

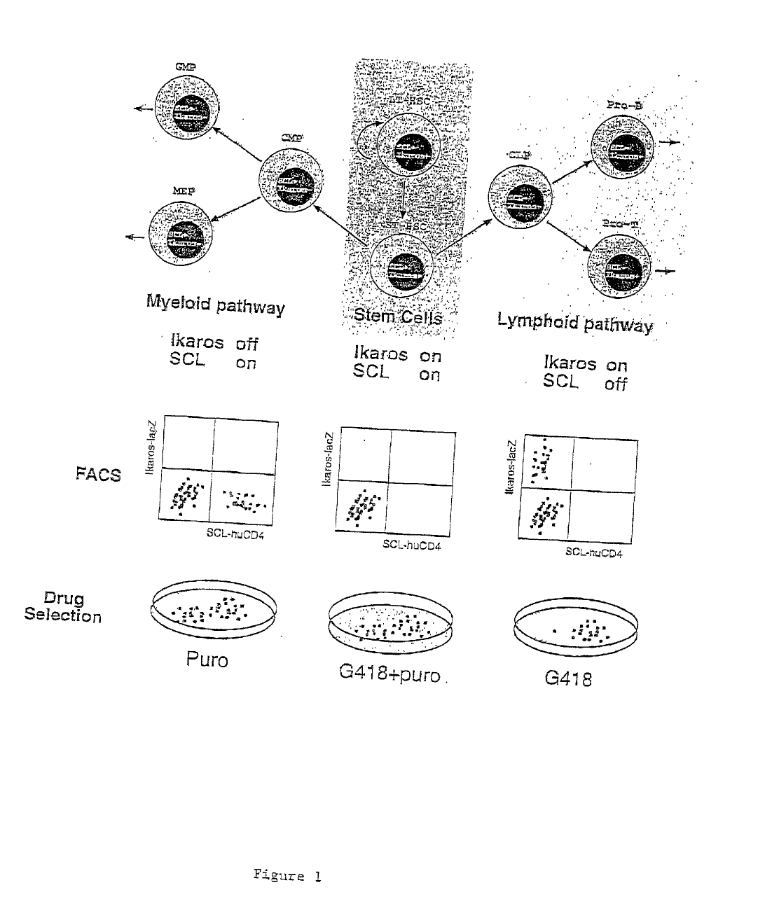

Hematopoietic stem cells give rise to progeny that progressively lose self-renewal capacity and became restricted to one lineage (Metcalf, D., Annuals of the New York Academy of Sciences, 872:289-203; discussion 303-4 (1999)). In the current model of major differentiation pathways from HSCs (FIG. 1), Long-term HSCs (LT-SHCs) give rise to Short-term HSCs (ST-HSCs). ST-HSCs then give rise to at least common lymphoid progenitors (CLPs) and common myeloid progenitors (CMPs), which can form all cells of the lymphoid lineages and of granulocyte/macrophage or megakaryocyte/erythrocyte lineages, respectively (Akashi, D., et al., Nature, 404:193-7 (2000); Weissman, I. L., Science, 287: 1442-6 (2000); Weissman, I. L., Cell, 100:157-68 (2000); Kondo, M., et al., Cell, 91:661-72 (1997)). Many transcription factors have been shown to play important functional roles in HSCs and lineage specification. Among those, SCL and Ikaros are two well-characterized transcription factors that are co-expressed in HSCs, but are not expressed in the same differentiated lineages (Begley, C. G., et al., Blood, 93:2760-70 (1999); Georgopoulos, K., Current Opinion in Immunology, 9:222-7 (1997)).

The helix-loop-helix transcription factor SCL was first identified as a partner in the (Morrison, S. J., et al., Cell, 88:287-98 (1997); Szilvassy, S. J., et al., Proceedings of the National Academy of Sciences of the United States of America, 87:8736-40 (1990)) translocation associated with T cell leukemia. Disruption of SCL completely blocks the early hematopoietic program (Begley, C. G., et al., Blood, 93:2760-70 (1999)). Much evidence from gene targeting experiments suggested that SCL is essential for both primitive and definitive hematopoiesis in mouse (Elefanty, A. G., et al., Blood, 94:3754-63 (1999); Shivdasani, R. A., et al., Nature, 373:432-4 (1995); Porcher, C., et al., Development, 126:4603-15 (1999)). Expression studies by mRNA or RT-PRC indicate that SCL selectively expressed in early myeloid, erythroid, megakaryocytic and mast cell lineages, and is tightly regulated during hematopoiesis (Visvader, J., et al., Trends in Biochemical Sciences, 16:330-3 (1991); Green, A. R., et al., EMBO Journal, 10:4153-8 (1991); Green, A. R., et al., Oncogene, 6:475-9 (1991)). Using a lacZ knock-in strategy, it has been shown that cells expressing SCL are enriched for 12 day spleen forming units and myeloid and erythroid colony-forming cells. The differentiated progeny of most lineages (except the erythroid) were negative for SCL expression (Elefanty, A. G., et al., Blood, 94:3754-63 (1999)).

The Ikaros gene encodes a family of early hematopoietic and lymphocyte restricted zinc-finger transcription factors, which are essential for lymphoid lineage specification. Eight splice variants, which have common N-terminal and C-terminal domains, were found in different lymphoid cell lines. Mice homozygous for a mutation in the Ikaros DNA-binding domain fail to generate mature T and B-lymphocytes as well as their early progenitors (Molnar, A., et al., Molecular & Cellular Biology, 14:8292-303 (1994)). In addition to the lymphocyte defects, mice homozygous for an Ikaros null mutation also display a >30-fold reduction in long-term repopulation units, whereas mice homozygous for an Ikaros dominant negative mutation have no measurable activity (Georgopoulos, E., et al, Cell, 79:143-56 (1994); Winandy, S., et al., Cell, 83:289-99 (1995); Nichogiannopoulou, A., et al., Journal of Experimental Medicine, 190:1201-14 (1999)). Moreover, different isoforms of Ikaros were detected in HSCs (Klug, C. A., et al., Proceedings of the National Academy of Sciences of the United States of America, 95:657-62 (1998)). This evidence suggested that the Ikaros family of DNA binding factors is critical for the activity of HSCs in mouse.

Based on the HSC differentiation model (FIG. 1) and the expression patterns of SCL and Ikaros, it is possible to use reporters driven by SCL and Ikaros promoter to specify HSCs. There are many advantages to mark HSCs with SCL and Ikaros. First, SCL and Ikaros have non-overlapping expression patterns in differentiated lineage. Second, SCL and Ikaros have important functional roles in HSC function. Moreover, SCL and Ikaros are critical for hematopoietic lineage commitment. All these properties make them very useful markers to monitor not only the HSCs but also the differentiated lineages.

2. Target Reporters Into SCL and Ikaros Loci Through Gene Targeting and BAC-mediated Transgenesis.

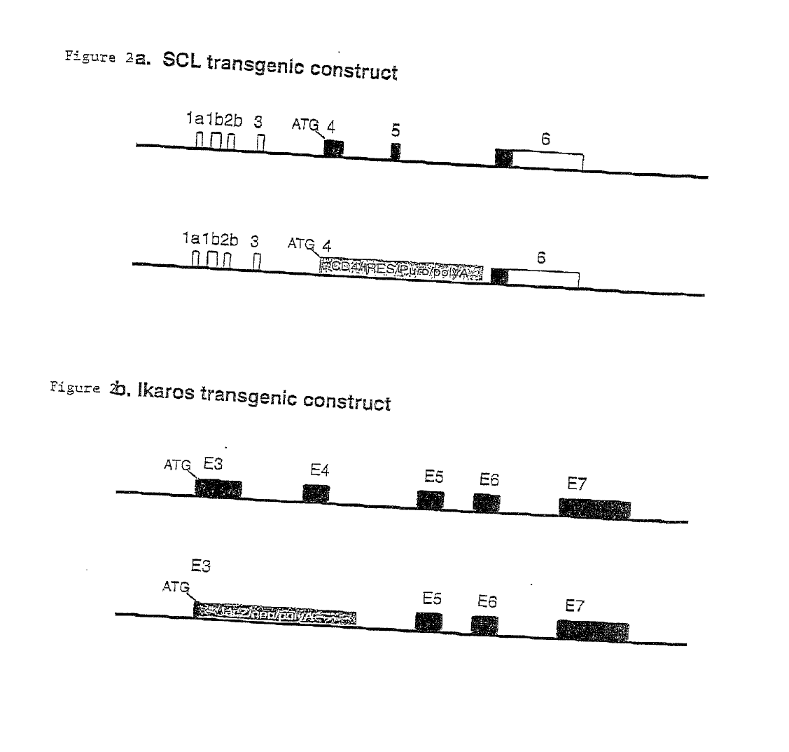

Although extensive promoter studies were carried out to identify HSC specific promoters from SCL and Ikaros, they had only limited success. Elements such as enhancers, locus control regions, and insulators, which are important for high level, tissue-specific, and integration site independent expression of transgene in mouse or Drosophila, may reside at a large distance (>50 kb) from the gene itself (Dillon, N., et al., Trends in Genetics, 9:134-7 (1993); Wilson, C., et al., Annual Review of Cell Biology, 6:679-714 (1990)). Conventional transgenic approaches using limited genomic DNA fragment (<20 Kb) frequently result in low-level transgene expression and extensive position effects. To avoid these problems and faithfully recapitulate the in vivo expression pattern of SCL and Ikaros with reporters, a BAC homologous recombination approach was used to target reporter sets into SCL and Ikaros loci (Yang, X. W., et al., Nature Genetics, 22:327-35 (1999); Yang, X. W., et al., Nature Biotechnology, 15:859-65 (1997)). HuCD4/IRES/puro was used as the reporter cassette to target SCL (FIG. 2a ), through which SCL positive cells can be stained by anti human CD4 antibody, or selected with puromycin in culture. BetaGeo (lacZneo) was used as a different reporter cassette to target Ikaros (FIG. 2b ), through which Ikaros positive cells can be stained by FDG, or selected with G418 in culture. Positive recombination constructs were isolated by PCR screening, and confirmed by southern-blot, then linearized and subjected to pronuclear injection. Transgenic lines from both constructs have been obtained; the animals can be bred, using known methods. The function of the reporters and their expression pattern as transgenes can be confirmed, using the transgenic animals, by histology and functional assay, such as Day 12 CFU-S assay and colongenic assays for hematopoietic progenitors (Metcalf, D., The hemtaopoietic cell stimulating factors, Amsterdam, The Netherlands, Elsevier (1984); Ploemacher, R. E. et al., Cell & Tissue Kinetics, 17:1-12 (1984); Ploemacher, R. E., et al., Cell & Tissue Kinetics, 17:375-85 (1984)). Verified SCL-huCD4/IRES/puro and Ikaros-BetaGeo founder lines can be further crossed to generate double transgenic mice.

3. Examine the Functional Properties of the Cells Marked by SCL and Ikaros.

Based on the HSC differentiation model (FIG. 1) and the expression profile of SCL and Ikaros, only the HSCs will express both reporters. However, using presently available methods, it has not been possible to isolate SCL/Ikaros positive cells from mice and to test their function. Therefore, it is essential to determine the percentage of HSCs by surface marker staining or Hochest staining phenotype that is positive for SCL/Ikaros expression, or conversely, the percentage of SCL/Ikaros positive cells that have an HSC surface antigen profile. In addition, it is important to determine the percentage of the SCL/Ikaros positive cells that are stem cells, as determined by a mice repopulating assay. These questions can be answered by carrying out the following experiments:

(i) Isolation of SCL/Ikaros positive cells from various organs of the transgenic mouse, including adult bone marrow, fetal liver, aorta-gonad-mesonephros (AGM) region and the yolk sac, followed by staining with antibodies against HSC surface markers or Hoechst 33342 dye (Akashi, K., et al., Nature, 404:193-7 (2000), Lemieux, M. E., et al., Blood, 86:1339-47 (1995); Szilvassy, S. J., et al., Blood, 74:930-9 (1989)). FACS will make it possible to quantify the percentage of SCL/Ikaros positive cells that have a HSCs surface marker profile. To verify their stem cell function, competitive repopulation assay are used to measure stem cell activity presented in SCL/Ikaros positive cells, and the nonoverlapping fraction between SCL/Ikaros positive cells and conventional HSC enrichment approaches. These experiments will reveal whether SCL/Ikaros and conventional HSC purification approaches can mark an overlapped HSC population, and whether HSC activity also exists in the non-overlapping fraction. If no stem cell activity is detected for the non-overlapping fraction, the cologenic activity of this fraction of cells can be further examined and experiments done to define the function of this fraction of cells.

(ii) Isolation of HSCs from those organs based on the HSC surface markers or Hochest 33342 staining (Akashi, K., et al., Nature, 404:193-7 (2000), Goodell, M. A., et al., Journal of Experimental Medicine, 183:1797-806 (1996); Lemieux, M. E., et al., Blood, 86:1339-47 (1995); Szilvassy, S. J., et al., Blood, 74:930-9 (1989)). Staining of the purified HSCs with anti human CD4 antibody or fluorescent lacZ substrate FDG. FACS sorting to quantify the percentage of HSCs by surface marker staining or Hochest 33342 staining that is also positive for SCL/Ikaros expression. In this way, it is possible to determine whether SCL/Ikaros can mark a more specific fraction of HSCs. If SCL/Ikaros can define a more specific fraction of HSCs, it will be interesting to find their stem cells activity in terms of short-term and long-term repopulation.

4. Utilizing Other HSC Specific Genes in Combination With SCL or Ikaros to Mark HSCs.

Conventional approaches could enrich HSCs to relative homogeneity with over 1000-fold enrichment (Szilvassy, S. J., et al., Blood, 74:930-9 (1989)). Although it has been shown that Day 12 CFC-S could be enriched by 50-100 fold by SCL alone (Elefanty, A. G., et al., Blood, 94:3754-63 (1999); Sanchez, M., et al., Development, 126:3891-904 (1999)), it is possible to use other known genes that play important functional roles in HSC function to mark HSCs in combination with SCL, or Ikaros. Dr. Skarness at U. C. Berkely has carried out a unique large-scale screen to identify ES clones in which membrane or secreted molecules are selectively trapped by betaGeo reporter (Skarnes, W. C., et al., Proceedings of the National Academy of Sciences of the United States of America, 92:6592-6 (1995); Brennan, J., et al., Methods in Molecular Biology, 97:123-38 (1999)). Among his collections of ES clones, some are trapped in known genes that play important functional roles in HSC function. Dr. Skarness has kindly provided the ES trapping cell lines Notch-1-betageo, and c-kit-betageo. These ES cells have been injected into blastocysts to generate mouse chimeras and chimeras for Notch-1-betageo have been obtained. As an alternative, these mice can be bred with SCL, or Ikaros transgenic mouse, and HSCs can be defined with different combinations of markers. If double transgenic markers fail to specify HSCs, three transgenes can be used to define HSCs.

5. Explore Conditions to Control HSCs Self-renewal and Lineage Commitment in vitro.

It is possible to test whether enriched HSCs can be selectively cultured in vitro in the presence of G418 and puromycin on a HSC supportive stromal cell line. Committed progenitors will be eliminated from the culture since they only express one of the two transgenic markers. Whether the SCL/Ikaros marker profile of HSC will change in the long-term culture (Moore, K. A., et al., Blood, 89:4337-47 (1997)) will be determined. The vast majority of cells in a HSC in vitro long-term culture are differentiated cells. Eliminating the differentiated cells makes it possible to determine whether their elimination removes the potential negative feedback released by HSC progeny. If removing differentiated cell results in expansion of SCL/Ikaros positive HSCs, this suggests that there is negative feedback from the differentiated cells. If this is not the case, it is also possible to test quantitative responses of HSCs to different exogenous signal stimulation, including combinations of growth factor and stromal cells. The expansion of HSCs with the SCL/Ikaros markers can be monitored by FACS (huCD4/lacZ). Mice repopulating assays can be used to verify whether the increase of the number of SCL/Ikaros positive cells indicates the proliferation of HSCs in activity. Since the assay is simple and fast, it is possible to systematically explore a lot of different conditions in a short time frame. In the past, surface markers have been used to monitor ex vivo expansion of HSCs. It has been observed that cells lost their pluripotency while retaining the surface marker profile, indicating that these markers are not necessary functionally correlated with HSC activity.

6. Retroviral Expression Cloning of Signal Molecules That Control HSC Self-renewal, Lineage Commitment

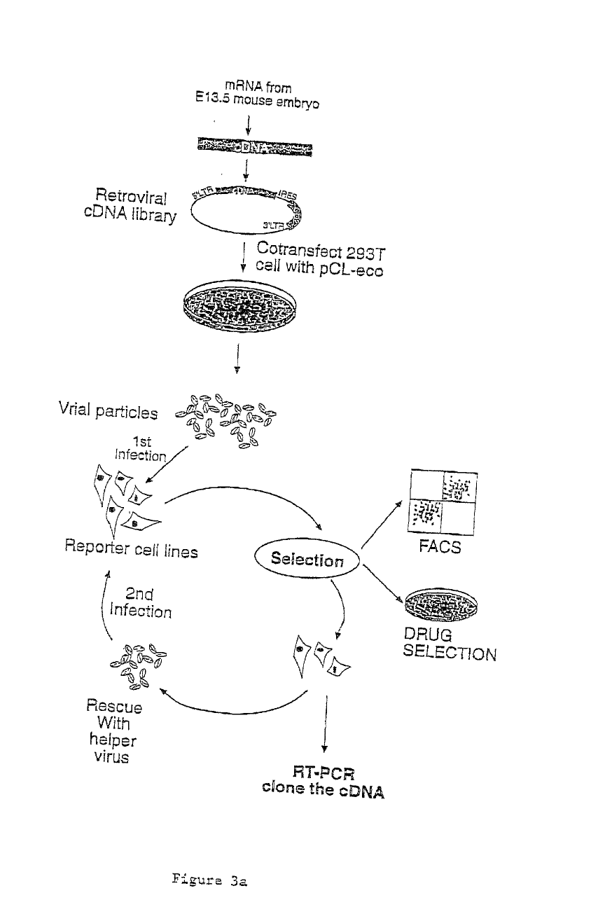

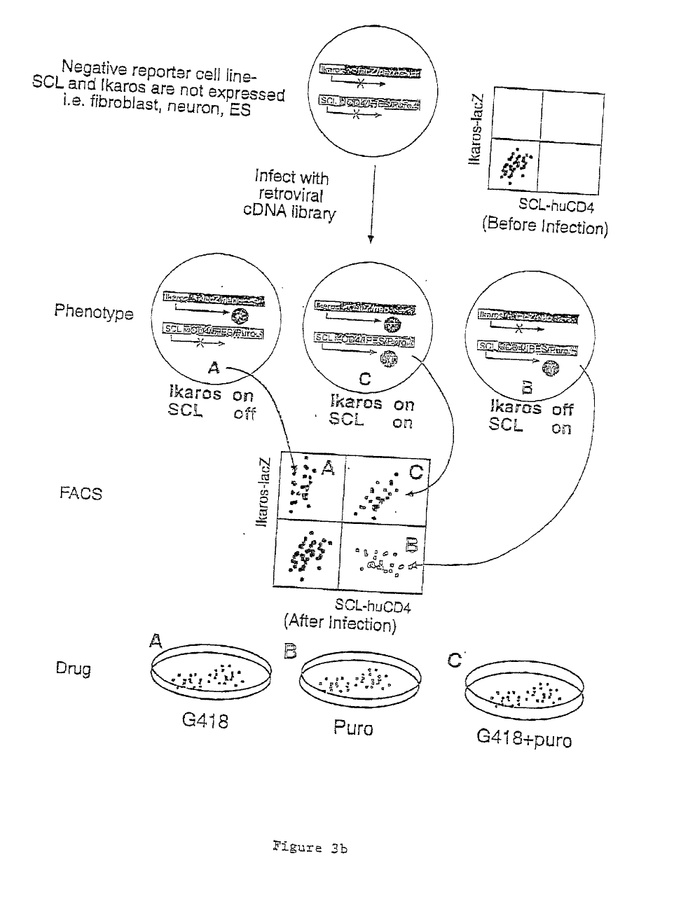

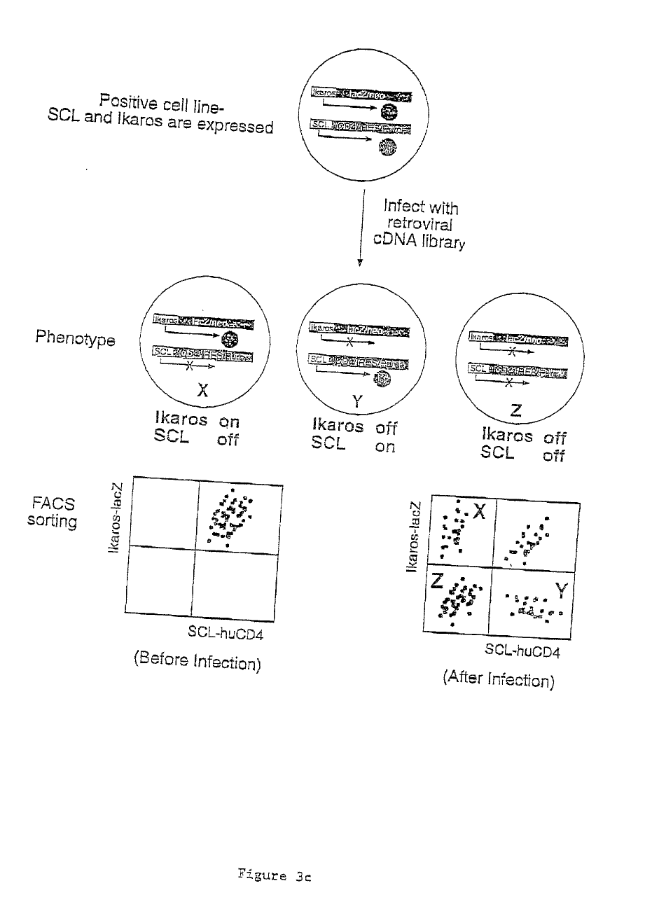

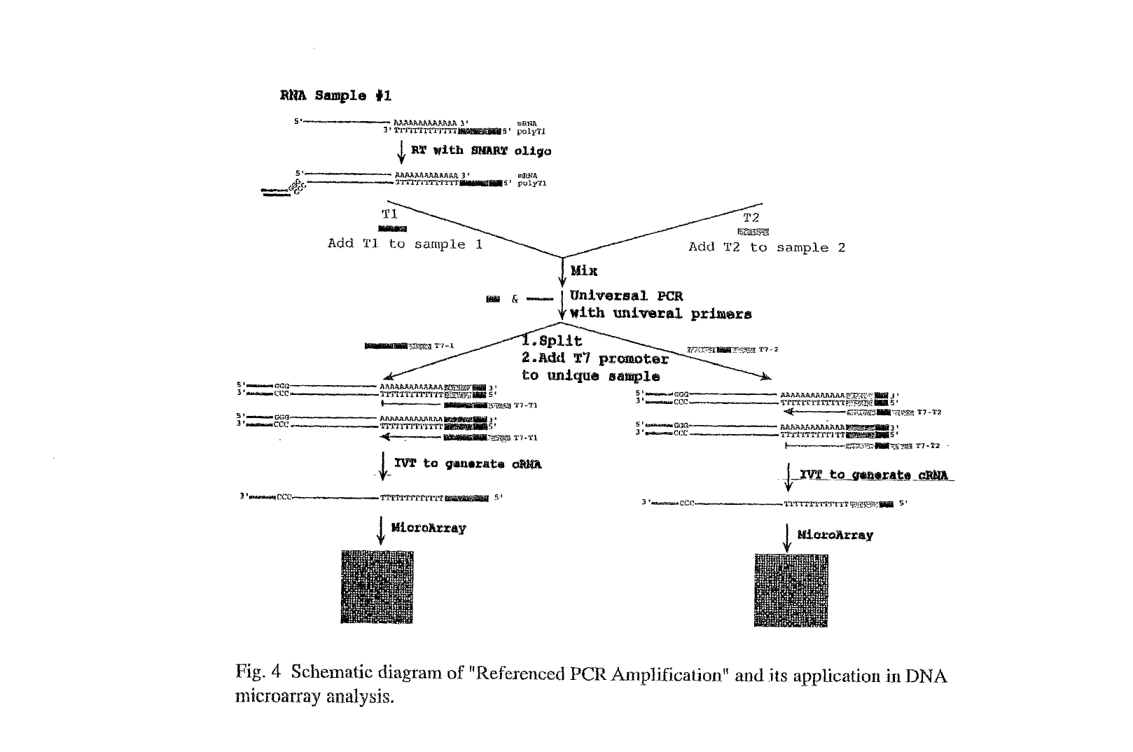

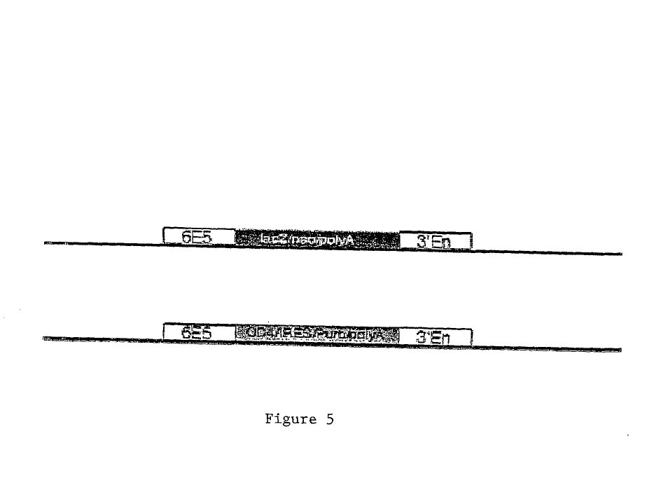

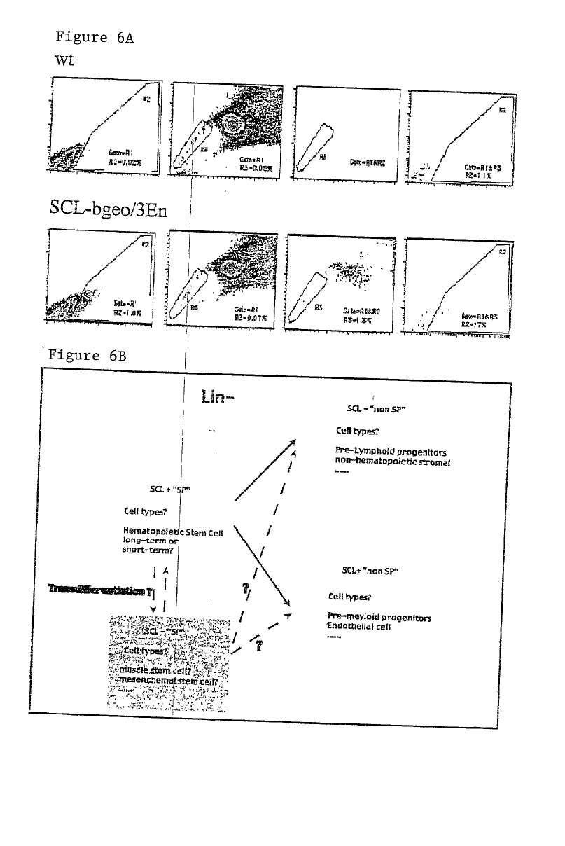

The cloning strategy is based on the assumption that over-expression of a functional or dominant negative regulator will change SCL/Ikaros expression in a reporter cell line (Liu, X., et al., Analytical Biochemistry, 280:20-8 (2000); Hua, X., et al., Genes & Development 12:3084-95 (1998)). The change of the SCL/Ikaros reporter expression can be readily detected by FACS sorting (huCD4, FDG) or selection with one or more drugs (G418, puro). By infecting a reporter cell line with a retroviral cDNA library, the cDNAs that cause the change of reporter expression can be readily identified. A retroviral cDNA library of 2.5 millions independent clones from E13.5 fetal liver has been produced. E13.5 fetal liver was chosen as the mRNA source because of the expansion of HSCs in fetal liver at day 12 and 16 of gestation (Ema, H., et al., Blood, 95:2284-8 (2000)). mRNA was isolated from E13.5 fetal liver, and cDNAs were synthesized. Then, the E13.5 fetal liver cDNAs were directionally cloned into a bicistronic retroviral vector MSCV-IRES-CD2 to generate the cDNA library. The IRES driven CD2 reporter in the vector can be used as a marker to detect infection efficiency and expression level of exogenous genes (Liu, X., et al., Analytical Biochemistry, 280:20-8 (2000); Hua, X., et al., Genes & Development 12:3084-95 (1998)). Viral particles were then packaged by co-transfection of the library cDNAs and pCL-eco into 293T or BOSC cells (Naviaux, R. K., et al., Journal of Virology, 70:5701-5 (1996)). By infecting reporter cell lines with the retroviral cDNA library, it will be possible to select infected reporter cells in which SCL or Ikaros expression patterns are changed by the correspondent integrated cDNA. The corresponding cDNA can be isolated by a rescue virus or RT-PCR. Many interesting questions can be studied with this system (FIG. 3a ).

Signal molecules that control SCL and Ikaros expression are likely to play important functional roles in HSC self-renewal or lineage commitment. From the double transgenic mice, it is possible to isolate various cell types that are positive or negative for the expression of the two markers, including HSCs that are positive for the expression of the two markers, embryonic fibroblast and ES cells that are negative for the expression of both markers. Introduction of a retroviral cDNA library into the above cell lines, makes it possible to identify intracellular signal molecules that turn on or turn off (1) only SCL expression, (2) only Ikaros expression, (3) both SCL and Ikaros expression. With proper selection approaches, these cDNAs can easily be isolated. Both positive and negative reporter cell lines can be used in the selection. Positive regulators for SCL and Ikaros expression can be isolated with a negative reporter cell line in which SCL/Ikaros are normally not expressed (FIG. 3b ). Conversely, negative regulators for SCL and Ikaros expression can be isolated with a positive reporter cell line in which SCL/Ikaros are normally expressed (FIG. 3c ).

7. Characterize the Cis-acting Regulatory DNA Regulatory Elements of Two Other Gene Required for HSC Differentiation and Maintenance, LMO2 and LYL1

Like SCL, LMO2 and LYL1 are both expressed in hematopoietic and endothelial cells during development; both gene loci are small (60 and 20 kb, respectively) thus facilitating comparative sequence analysis; and both have important biological connections with SCL. LMO2 encodes a lim domain transcriptional cofactor with several connections to SCL. LMO2 is coexpressed with SCL in endothelium and blood. Like SCL, LMO2 was originally identified as a T-cell oncogene. LMO2 and SCL proteins form part of a multiprotein complex both in normal and malignant cells. The knockout phenotypes for SCL and LMO2 are very similar with complete absence of hematopoiesis and defects in yolk sac angiogenesis indicating critical functions for multiprotein complexes containing SCL/LMO2. LYL1 is a paralogue of SCL with more than 90% sequence identity in the bHLH region and was also originally identified as a T-cell oncogene. Like SCL, LYL1 is expressed in endothelial and blood cells during murine embryonic development, and in cell lines representing hematopoietic progenitors. Some aspects of their shared expression pattern may be due to conserved regulatory circuits, which were already acting on the common ancestral gene of both SCL and LYL1. Comparison of LMO2, LYL1 and SCL enhancers that target blood and/or endothelium will therefore begin to elucidate the way in which regulatory information important for blood/endothelial development is encoded in the primary DNA sequences.

Relevance and Significance

Genetically marking HSCs by targeting SCL and Ikaros provides a powerful tool to detect, monitor, and select HSCs in real-time. This will overcome the limitation of the current functional assays for HSCs that are quite tedious and time-consuming, and cannot be used in real-time to monitor HSC activity. This new tool will also make it possible to gain more insight into the molecular mechanisms that control HSC self-renewal and lineage commitment. Any progress in this direction is not only biologically significant, but also has implications in bone marrow transplantation, gene therapy, and disease pathogenesis (e.g., leukemia). In addition, SCL and Ikaros play important functional roles in HSC function and lineage commitment. Aberrant expression of SCL and Ikaros in vivo causes many blood diseases including certain type of leukemia and lymphoma. Understanding the signals that control SCL and Ikaros expression will provide insights into the mechanism of these diseases.

While this invention has been particularly shown and described with references to preferred embodiments thereof, it will be understood by those skilled in the art that various changes in form and details may be made therein without departing from the scope of the invention encompassed by the appended claims.

What is claimed is:

1. A method of identifying hematopoietic stem cells, comprising
a) targeting two different reporter DNAs into two different functionally important genomic loci of hematopoietic stem cells in such a manner that expression of the reporter DNA is driven by the promoter of the genomic locus into which the reporter DNA is targeted, thereby producing a population comprising successfully targeted hematopoietic stem cells and other cells; and
b) subjecting the population of cells produced in (a) to conditions under which successfully targeted hematopoietic stem cells survive and the other cells in the population do not survive, thereby identifying hematopoietic stem cells.

2. The method of claim 1, wherein the hematopoietic stem cells are mouse cells or human cells.

3. The method of claim 1 wherein the two different functionally important genomic loci of hematopoietic stem cells are the Stem Cell Leukemia (SCL) locus and the Ikaros locus.

4. The method of claim 1 further comprising c) isolating the hematopoietic stem cells.

5. A method of identifying hematopoietic stem cells, comprising
a) targeting a first reporter DNA into a Stem Cell Leukemia (SCL) locus and a second reporter DNA which is different from the first reporter DNA into an Ikaros locus of hematopoietic stem cells in such a manner that expression of the first reporter DNA is driven by the promoter of the SCL locus and expression of the second reporter DNA is driven by the promoter of the Ikaros locus, thereby producing a population comprising successfully targeted hematopoietic stem cells and other cells; and
b) subjecting the population of cells produced in (a) to conditions under which successfully targeted hematopoietic stem cells survive and the other cells in the population do not survive, thereby identifying hematopoietic stem cells.

6. The method of claim 5, wherein the hematopoietic stem cells are mouse cells or human cells.

7. The method of claim 5 further comprising c) isolating the hematopoietic stem cells.

8. The method of claim 5 wherein the first reporter DNA is a huCD4/IRES/puro construct and the second reporter DNA is a βneo(lacZneo) construct.

9. Isolated hematopoietic stem cells produced by the method of claim 4.

10. Isolated hematopoietic stem cells produced by the method of claim 7.

11. Isolated hematopoietic stem cells comprising two different reporter DNAs which are present in two different functionally important genomic loci of the hematopoietic stem cells, wherein expression of the reporter DNAs is driven by the promoters of the genomic loci into which the reporter DNAs are targeted.

12. The isolated hematopoietic stem cells of claim 12, wherein the hematopoietic stem cells are mouse cells or human cells.

13. The isolated hematopoietic stem cells of claim 12 wherein the two different functionally important genomic loci of hematopoietic stem cells are the Stem Cell Leukemia (SCL) locus and the Ikaros locus.

14. Isolated hematopoietic stem cells comprising a first reporter DNA which is present in a Stem Cell Leukemia (SCL) genomic locus of the hematopoietic stem cells and a second reporter DNA which is different from the first reporter DNA and which is present in an Ikaros genomic locus of the hematopoietic stem cells, wherein expression of the first reporter DNA is driven by the promoter of the SCL locus and expression of the second reporter DNA is driven by the promoter of the Ikaros locus.

15. The isolated hematopoietic stem cells of claim 14, wherein the hematopoietic stem cells are mouse cells or human cells.

16. The isolated hematopoietic stem cells of claim 14 wherein the first reporter DNA is a huCD4/IRES/puro construct and the second reporter DNA is a βneo(lacZneo) construct.
